(19) United States
(12) Patent Application Publication
(10) Pub. No.: US 2005/0175967 A1
(43) Pub. Date: Aug. 11, 2005

(54) Detecting dental apical foramina

(75) Inventors: Naim Karazivan, Laval, QC (CA); Emmanuel Montini, Ste-Anne-des-Lacs (CA); Francois Ayotte, Laval (CA)

Correspondence Address: GOUDREAU GAGE DUBUC, 800 PLACE VICTORIA, SUITE 3400, MONTREAL, QUEBEC, H4Z 1E9, CA

(73) Assignee: NEKS Technologies Inc., Laval, QC (CA)

(21) Appl. No.: 10/513,578

(22) PCT Filed: May 9, 2003
(86) PCT No.: PCT/CA03/00652

(30) Foreign Application Priority Data
May 9, 2002 (CA) 2,385,527

(51) Int. Cl. A61B 6/00; A61C 1/00

(52) U.S. Cl. 433/229; 600/473

(57) ABSTRACT

A system for detecting dental root canal apical foramina of a tooth (T) comprises an electromagnetic conductor for directing at least one initial radiation (Ir) onto a tooth structure to be evaluated, an electromagnetic collector for collecting at least one resulting electromagnetic radiation (Rr) that has been at least one of reflected by and transmitted through the tooth (T) as a result of the initial radiation (Ir). The collector is adapted to deliver the resulting electromagnetic radiation (Rr) to a detection device (D). The detection device (D) is adapted to compare at least one intensity of the at least one resulting radiation (Rr) with at least one predetermined value that corresponds to one of the presence and absence of dental root canal apical foramina. This enables the diagnosis of the presence or absence of dental root canal apical foramina on the tooth structure.

### BACKGROUND OF THE INVENTION

1. Field of the Invention

The present relates to the detection of dental root canal apical foramina and, more particularly, to a system and method for detecting dental root canal apical foramina.

2. Description of the Prior Art

Detecting the root canal apical foramina is a crucial part in root canal treatment.

Tactile sense can be used to find the apical foramina, but this technique is unreliable when used alone.

Currently, the most widely used technique is based on X-ray pictures taken with a radio-opaque instrument in the root canal. Another instrument currently giving an approximate idea of the root canal position is the electric apex locator.

More recently, new techniques have started to appear, such as a fiber-optic endodontic apparatus and method (see U.S. Pat. No. 5,503,559, 1996). These apparatus and method were devised to localise the root canal apical foramina, to help tissues recognition inside the canal and to cure radiation-curable cement inside the canal.

### SUMMARY OF THE INVENTION

It is therefore an aim of the present invention to provide a novel system for the detection of dental root canal apical foramina.

It is also an aim of the present invention to provide a novel system for the detection of dental root canal apical foramina, which automatically detects the root canal apical foramina based on the intensity of the reflected wavelengths and/or on spectral reflectance characteristics of the root canal apical foramina.

It is a further aim of the present invention to provide a system in which a visual, sound-based, or other, signal is given following detection of dental root canal apical foramina, wherein this detection results from measurements made on the tooth and taken in one or more predetermined ranges of wavelengths that are appropriate for discriminating the spectral reflectance characteristics that constitute a signature of the presence of root canal apical foramina, and/or on the intensity of the reflected wavelengths.

Therefore, in accordance with the present invention, there is provided a system for locating a root canal apical foramen of a tooth, comprising a conductor for bringing at least one initial radiation onto a root canal, a collector for collecting at least one resulting radiation that has been reflected by and/or transmitted through the root canal as a result of said initial radiation, said collector being adapted to deliver said resulting radiation to a detection device, said detection device being adapted to compare at least one intensity of said at least one resulting radiation with at least one predetermined value that corresponds to the presence or absence of a root canal apical foramen, thereby enabling to locate the root canal apical foramen.

Also in accordance with the present invention, there is provided a system for locating a root canal apical foramen of a tooth, comprising a conductor for directing at least one initial radiation onto a root canal, a collector for collecting at least one resulting radiation that has been reflected by and/or transmitted through the root canal as a result of said initial radiation, said collector being adapted to deliver said resulting radiation to a detection device, said detection device being adapted to compare at least one wavelength of said at least one resulting radiation with at least one predetermined value that corresponds to the presence or to the absence of a root canal apical foramen, thereby enabling to locate the root canal apical foramen of the tooth.

Further in accordance with the present invention, there is provided a method for locating a root canal apical foramen of a tooth, comprising the steps of irradiating a root canal of a tooth with an initial radiation, collecting a reflected and/or transmitted resulting radiation, comparing the wavelength(s) and/or the intensity(ies) of radiation(s) with a predetermined value(s) that corresponds to the presence or absence of a root canal apical foramen, thereby enabling to locate the root canal apical foramen.

Still further in accordance with the present invention, there is provided a system for locating a root canal apical foramen of a tooth, comprising a probe adapted to be displaced in a root canal of a tooth, illumination means for illuminating with an incident light a region of the root canal, detection means for collecting the resulting light reflected by and/or transmitted through the root canal, and an analyzing system for providing a signal when measurements on the resulting light in one or more predetermined ranges of wavelengths fall within any first predetermined range of values that are characteristic of dental root canal apical foramen, or when said measurements do not fall within any second predetermined range of values that are characteristic of artifacts other than root canal apical foramen.

Still further in accordance with the present invention, there is provided a system for locating a root canal apical foramen of a tooth, comprising a probe adapted to be displaced in a root canal of a tooth, illumination means for illuminating with an incident light a region of the root canal, detection means for collecting the resulting light reflected by and/or transmitted through the root canal, and an analyzing system for providing a signal when intensity measurements on the resulting light indicate one of the presence and absence of a root canal apical foramen.

Still further in accordance with the present invention, there is provided a method for locating a root canal apical foramen of a tooth, comprising the steps of: (a) providing an incident light on a region of a root canal of a tooth; (b) collecting and measuring the resulting light reflected by and/or transmitted through said region of the root canal; (c) analyzing said resulting light to determine if said resulting light is representative of the presence of a root canal apical foramen; and (d) providing a signal to an operator that indicates that one of presence and absence of a root canal apical foramen has been detected in step (c).

### BRIEF DESCRIPTION OF THE DRAWINGS

Having thus generally described the nature of the invention, reference will now be made to the accompanying drawings, showing by way of illustration a preferred embodiment thereof, and in which:

FIGS. 1 and 4 are schematic diagrams of a system for the detection of dental root canal apical foramina in accordance with a first embodiment of the present invention;

FIGS. 2 and 5 are a schematic diagrams of a system for the detection of dental root canal apical foramina in accordance with a second embodiment of the present invention; and

FIGS. 3 and 6 are schematic diagrams of a system for the detection of dental root canal apical foramina in accordance with a third embodiment of the present invention.

### DESCRIPTION OF THE PREFERRED EMBODIMENTS

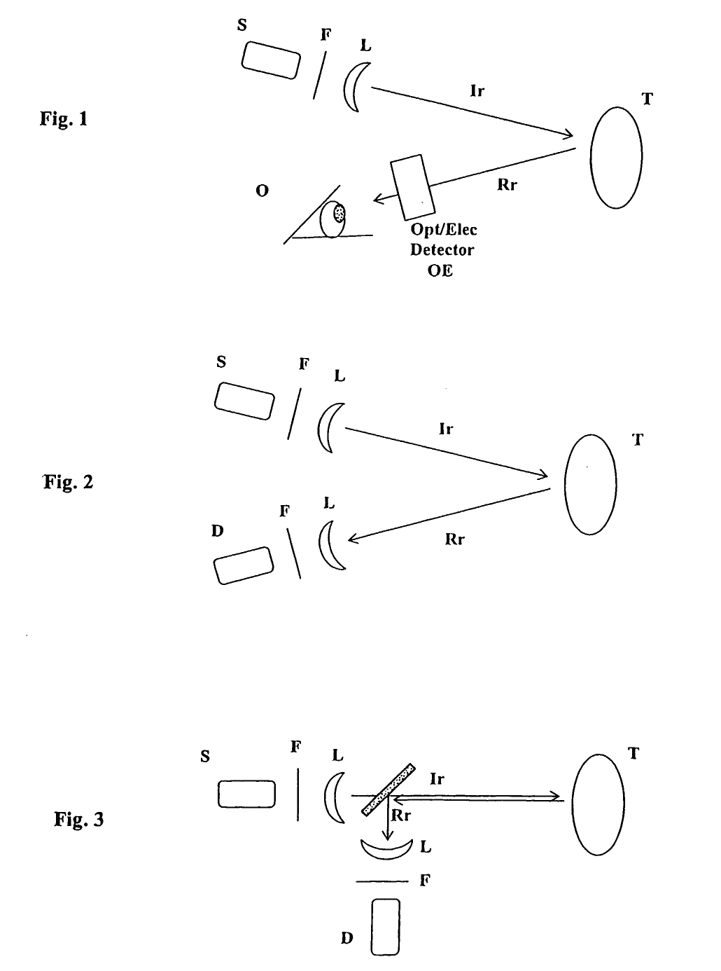

In accordance with the present invention, FIGS. 1 to 6 illustrate three systems for the automated location of root canal apical foramina in a patient's teeth.

Each system basically comprises three main mechanisms, that is (1) an optical hand-held tool (a buccal probe) for directing an incident light onto a tooth T and for capting light reflected by and/or light transmitted through the tooth T, (2) a device (e.g. a casing containing optical components, light sources, and acquisition and signal processing electronics) for providing a light source to the tool and for analyzing the reflected/transmitted light and providing a signal to an operator indicative of the presence of root canal apical foramina, and (3) a transmission device (e.g. a cable strand that includes optical fibres) that connects the hand-held tool to the analyzing device (casing). Some further details of these components can be found in PCT Publication No. WO01/23767 (based on PCT Application No. PCT/CA01/00063).

The systems of the present invention locate root canal apical foramina by analyzing the intensity of the reflected/transmitted light and/or by analyzing wavelengths of the reflected/transmitted light such as to discriminate the root canal apical foramina present in the root canal of a tooth from other tooth structures and from outside artifacts (e.g. gums, blood), and in fact from any artefact other than caries that the tool may encounter when it is directed inside the root canal.

The device described herein is suitable to detect root canal apical foramina.

The device described relies on the recognition of the reflectance and/or transmittance properties of structures related with root canal apical foramina from structures unrelated to root canal apical foramina when these structures are irradiated with visible or invisible ultra-violet (UV) or invisible infrared (IR) wavelength(s) radiation(s).

The invention is a root canal apical foramina detector principally based on a spectroscopic evaluation system of the reflectance and or transmittance properties of dental structures. When a structure is irradiated with an initial radiation(s) Ir, the radiation can in part be reflected on the structure surface and in part penetrate and travel inside this structure where some or all of those penetrating radiations can be deviated and/or reflected. Depending of the composition and/or shape and/or on the surrounding structures present, a specific structure can reflect and transmit a specific radiation differently than another structure. Depending on the origin of the irradiation and on the geographical position of the observation point for the resulting radiation(s) Rr (also called hereinafter “collected radiation”), the transmission and reflection will be different for a same structure.

In the present invention, initial electromagnetic radiation is brought to the root canal structure using an electromagnetic conductor coupled with a source. For example, an infrared electromagnetic radiation of around 860 nm can be used alone or with an electromagnetic radiation of around 630 nm. Any other suitable radiation or group of two or more radiations in the UV, visible or IR spectrum can be used.

The source S is an electromagnetic radiation generator (for all or parts of UV-Visible-IR). Multiple sources S can be used to obtain the desired radiation(s). Filter(s) F or other optical means can be used to obtain the desire radiation(s). For example, a visible radiation (i.e. visible light) of around 630 nm wavelength can be combined with an infrared radiation of around 860 nm wavelength.

Examples of sources S that can be used are: LEDs, laser-diodes, lasers, halogens light, neon light, or any other suitable type of emitting radiation source.

The spectral band(s) and the intensity(ies) of the radiation(s) to be generated by the source(s) S is (are) selected based on the characteristic that (those) radiation(s) has(have) a different behaviour when it (they) encounters directly or indirectly an apical structure outside the dental root canal than when it (they) encounters an apical structure inside the root canal structure. The wavelength(s) and the intensity(ies) is (are) selected when the more unambiguous distinction, with a determined configuration of the invention (collector, conductor, detection means, etc.), can be made between inside root canal structures and apical structures outside the dental root canal.

The initial radiation(s) Ir intensity is equal to the source S intensity less the lost in the conductor. The conductor brings the radiation from the source S to the structure to be evaluated.

A feedback system can be implemented to measure the initial radiation.

The conductor can be an optical fibre or bundle of optical fibres similar for example to some of the optical fibres used for laser-based endodontic treatment, or any other material suitable for radiation transmission. For example, the conductor can be made with lens(es) and/or mirror(s).

Lenses L, mirrors or other suitable optical means, can be inserted between the conductor and the source S to enhance the coupling of the radiation into the conductor.

The initial radiation(s) Ir can be modulated and synchronized with the detector to ease the recognition of the reflected/transmitted resulting radiation(s) Rr from this initial radiation(s) Ir from other radiation(s) resulting from another initial radiation(s) Ir or from noises. This method is sometimes called “Lock-in system”. One advantage of the lock-in system is its sensitivity even with very weak levels of radiation.

The resulting radiation(s) Rr is collected via a collector that brings the resulting radiation(s) Rr to a detection device D. The collector can be an optical fibre or a bundle of optical fibres or any other means suitable to bring the resulting radiation(s) Rr from the root canal inside the tooth T to the detection device D. For example, the collector can be made with mirrors and/or lenses.

The detection device D is used to compare the resulting radiation(s) Rr to at least one or part of one of the following measurements: other resulting radiation(s) Rr (i.e. resulting from irradiation effected at other times), noise(s) included in the resulting radiation(s) Rr or the initial radiation(s) Ir directly or indirectly, punctually or with variable of time or by using a function of this (those) measurement(s) with a predetermined range of value(s) corresponding to apical structures outside the dental root canal. For example, comparing the resulting radiation(s) Rr less the noise in that resulting radiation(s) Rr to a predetermined range of values that are in relation with the initial radiation, is a typical way of determining if the measurements correspond, or not, to apical structures outside the dental root canal: when using a specific Ir intensity, if Rr less noise in Rr is over a certain value that is in function of Ir, then the detection of root canal apical foramina is positive.

Another example is when an initial radiation(s) Ir with a wavelength around 860 nm is used. The resulting radiation(s) Rr then has a different intensity when it is in the root canal than when it is outside the root canal. When the resulting radiation(s) Rr is higher or lower than a certain value, which is in relation with the initial radiation(s) Ir intensity, the diagnosis of root canal apical foramina is positive.

This detection device D can be made with a semi-conductor detector (e.g. photo-diode or LCD) that converts the resulting radiation(s) Rr into a signal or a plurality of signals. This detector sends this (those) signal(s) to an electronic or electro-mechanic system EAM that analyses the signals so as to determine if there is presence of root canal apical foramina, or not. A stimulus(i) Stim (e.g. sound, light, vibration, etc.), is then generated through this electronic system EAM to inform the operator O. An analog converter C/A is provided upstream of the stimulus Stim in the second and third embodiments of FIGS. 3 to 6.

The detection mean D can also include a semi conductor radiation detector (e.g. photo-diode) connected to a system that converts at least one or a part of one of the following measurements: other resulting radiation(s) Rr (i.e. resulting from irradiation effected at other times), noise(s) included in the resulting radiation(s) Rr or the initial radiation(s) Ir directly or indirectly, punctually or with variable of time or by using a function of the (those) measurement(s) into the corresponding stimulus(i) Stim to the operator O (e.g. variable sound intensity, variable light signal, etc.). The operator O then makes the distinction between stimuli associated with root canal apical foramina and stimuli associated with root canal structure. For example, the detection device can be a sound generator that gives a sound intensity equivalent to the intensity of the resulting radiation. Another example can be a graphical screen display of different radiation measurements and where the Operator O uses his judgment to identify the location of the root canal apical foramina.

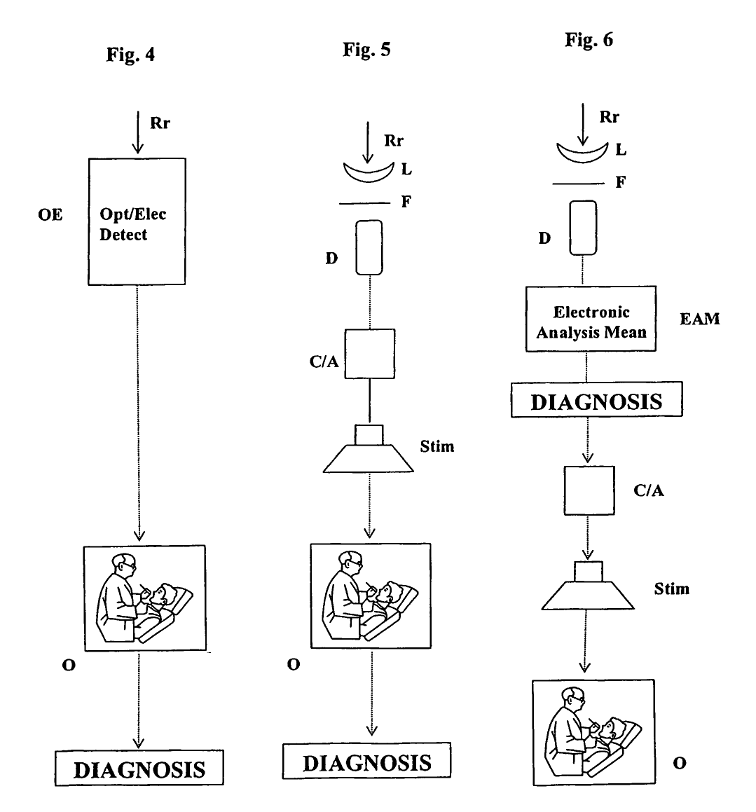

The detection device OE of FIGS. 1 and 4 can be made with physical means that convert at least one or a part of one of the following measurements on: the resulting radiation(s) Rr, noise(s) included in the resulting radiation(s) Rr or the initial radiation(s) Ir directly or indirectly, punctually or with variable of time or by using a function of the (those) measurement(s) into a corresponding stimuli to the operator O (e.g. sound, light signal, etc.), who again then makes the distinction between stimuli associate with root canal apical foramina and stimuli associated with root canal structure. For example, this detection device OE can be a mirror that reflects the resulting radiation(s) Rr to the operator O.

For example, the electronic analysis system EAM could be made of an electronic processor and an algorithm based on independent functions of the two demodulated signals received, if the initial radiations Ir of 860 nm and 630 nm are used in conjunction with a lock-in system.

It is noted that the source S can be modulated in intensity and/or in wavelength. By modulating the current and/or the voltage of a source, the intensity and the range of wavelengths can change and the constant variation in the radiation can be used instead of multiple sources.

To enhance the detection, it may be desirable to characterize the typical response radiation on different structures in the mouth of some patients prior to beginning detection of root canal apical foramina.

The detection system can be partially or totally included in a device for root canal preparation (e.g. rotative handpieces, ultrasonic/sonic devices, etc.).

Also, the detection system can be designed to work in conjunction with an instrument used for root canal treatment.

The components of the present systems that will be put in contact with intra-oral tissues can be made sterilizable.

The systems of the present invention can comprise multiple conductors that can bring the initial radiation(s) from different angles or regions so as to possibly enable focalizing radiation on three dimensional regions or enable obtaining multiple readings on the same region.

A drying device can be incorporated in the systems of the invention for reducing the number of contaminants between the viewing tip of the instrument and the root canal.

An intermediate substance can be inserted between the viewing end of the conductor, or the collector, and the root canal to minimise undesired reflections and/or to act as a filter. For example, a transparent gel-like substance could be used.

The present systems may comprise a special marker having an affinity with root canal apical foramina and special reflectance/transmittance radiation property(ies) that can enhance or enable the detection of root canal apical foramina. For instance, a blue marker that has an affinity with root canal apical foramina will reflect radiation(s) wavelength(s) corresponding to blue.

A combined sonic or ultrasonic generator or stress generator can be implemented to induce stress or oscillation or movement in root canal enabling interferences and revealing weaker structure.

Teeth have a large morphologic variability that induces a high variability in optical response. For that reason, a comparative method can be implemented to enable the optical response to be standardized. By changing the position of the emitting point and the angle of emission, recorded value can be compared.

A liquid (e.g. water) delivery system can be incorporated to the invention to enable cleaning and/or obtaining an optical medium between the viewing end of the conductor or the collector and the root canal.

The invention can comprise, before the collector, a perforated component that enables only radiation that is parallel to the axis of this perforated component to enter the collector. This perforated component can enable the determination of the origin of the radiation ray. For example, this perforated component can enable to determine from which three dimensional area does the radiation come.

The collector can be made with a bundle of optical fibres. This coherent bundle enables the analysis region-by-region of the coming radiation. This region-by-region analysis can be obtained by using a plurality of semi-conductor detectors or by using an opaque pattern.

The invention can comprise a mean to archive data. For example, the invention can be connected to a computer that can save the data for later use.

The probe end (i.e. the distal end of the tool that faces the root canal and that contains the collector and/or the conductor) may comprise graduation marks to facilitate positioning.

The systems of the present invention can also include some recalibration and/or self-testing functions. For example, if optical fibres are used, it is possible to verify if the fibres are too worn out to be efficiently used and should thus be replaced by testing the intensity of a reference light that passed through the fibres.

Also, as the spectral responses of various artifacts other than root canal apical foramina are known, such as those of blood and gums, the apical foramina can be either directly detected or indirectly detected as the detection can be made to either detect its presence or its absence (i.e. the presence of a non-apical foramina artifact).

The detection of the end of the canal is difficult because the insertion of a fibre in a very small space forces the use of a very fine optical fibre (25 microns), which can only convey a small amount of energy and which is very small with respect to debris (healthy or decomposing nerve, blood micro-vessel) that are present in the canal.

Alternatively, the end of the canal could be detected without inserting the fibre up to the end of the tooth.

Another technique would reside in conveying a large quantity of light (one or more laser(s), red or infrared) from the occlusal, and in collecting, via a very thin fibre that can be inserted to the end of the canal, light returning from the occlusal.

An alternative technique would use an emitting fibre capable of being inserted to the end of the canal of the tooth and a large collecting fibre in a more occlusal location to collect the signal.

1. A system for locating a root canal apical foramen of a tooth, comprising a conductor for bringing at least one initial radiation onto a root canal, a collector for collecting at least one resulting radiation that has been reflected by and/or transmitted through the root canal as a result of said initial radiation, said collector being adapted to deliver said resulting radiation to a detection device, said detection device being adapted to compare at least one intensity of said at least one resulting radiation with at least one predetermined value that corresponds to the presence or absence of a root canal apical foramen, thereby enabling to locate the root canal apical foramen.

2. A system as defined in claim 1, wherein at least one wavelength of said at least one initial radiation is in the infrared spectrum.

3. A system as defined in claim 1, wherein a marker with an affinity with root canal apical foramina is provided, said marker having particular reflectance/transmittance properties when irradiated with visible or invisible radiation.

4. A system as defined in claim 1, wherein at least one of said conductor and said collector comprises an optical fibre adapted to be inserted in the root canal.

5. A system as defined in claim 4, wherein said optical fibre is contained in a tube adapted to be inserted in the root canal.

6. A system as defined in claim 1, wherein said detection device is also adapted to compare at least one wavelength of said at least one resulting radiation with at least one further predetermined value that corresponds to one of the presence and absence of dental root canal apical foramina.

7. A system as defined in claim 1, wherein said detection device is adapted to provide a signal that indicates one of the presence and absence of dental root canal apical foramina.

8. A system as defined in claim 1, wherein said detection device is adapted to provide information that is indicative of one of the presence and absence of dental root canal apical foramina, such that said information can be interpreted by an operator that is then able to locate the root canal apical foramina.

9. A system for locating a root canal apical foramen of a tooth, comprising a conductor for directing at least one initial radiation onto a root canal, a collector for collecting at least one resulting radiation that has been reflected by and/or transmitted through the root canal as a result of said initial radiation, said collector being adapted to deliver said resulting radiation to a detection device, said detection device being adapted to compare at least one wavelength of said at least one resulting radiation with at least one predetermined value that corresponds to the presence or to the absence of a root canal apical foramen, thereby enabling to locate the root canal apical foramen of the tooth.

10. A method for locating a root canal apical foramen of a tooth, comprising the steps of irradiating a root canal of a tooth with an initial radiation, collecting a reflected and/or transmitted resulting radiation, comparing the wavelength(s) and/or the intensity(ies) of radiation(s) with a predetermined value(s) that corresponds to the presence or absence of a root canal apical foramen, thereby enabling to locate the root canal apical foramen.

11. A method as defined in claim 10, wherein at least one wavelength of said initial radiation is in the infrared spectrum.

12. A method as defined in claim 10, wherein a marker with an affinity with root canal apical foramina is provided, said marker having particular reflectance/transmittance properties when irradiated with visible or invisible radiation.

13. A method as defined in claim 10, wherein said initial radiation and said resulting radiation are conveyed via at least one optical fibre adapted to be inserted in the root canal.

14. A method as defined in claim 13, wherein said optical fibre is contained in a tube adapted to be inserted in the root canal.

15. A system for locating a root canal apical foramen of a tooth, comprising a probe adapted to be displaced in a root canal of a tooth, illumination means for illuminating with an incident light a region of the root canal, detection means for collecting the resulting light reflected by and/or transmitted through the root canal, and an analyzing system for providing a signal when measurements on the resulting light in one or more predetermined ranges of wavelengths fall within any first predetermined range of values that are characteristic of dental root canal apical foramen, or when said measurements do not fall within any second predetermined range of values that are characteristic of artifacts other than root canal apical foramen.

16. A system for locating a root canal apical foramen of a tooth, comprising a probe adapted to be displaced in a root canal of a tooth, illumination means for illuminating with an incident light a region of the root canal, detection means for collecting the resulting light reflected by and/or transmitted through the root canal, and an analyzing system for providing a signal when intensity measurements on the resulting light indicate one of the presence and absence of a root canal apical foramen.

17. A method for locating a root canal apical foramen of a tooth, comprising the steps of: (a) providing an incident light on a region of a root canal of a tooth; (b) collecting and measuring the resulting light reflected by and/or transmitted through said region of the root canal; (c) analyzing said resulting light to determine if said resulting light is representative of the presence of a root canal apical foramen; and (d) providing a signal to an operator that indicates that one of presence and absence of a root canal apical foramen has been detected in step (c).

18. A method as defined in claim 17, wherein, in step (c), the intensity of said resulting light is measured to evaluate the presence or absence of root canal apical foramina.
